(19) United States
(12) Patent Application Publication
(10) Pub. No.: US 2006/0282124 A1
(43) Pub. Date: Dec. 14, 2006

(54) Apparatus and method for cardioversion

(76) Inventors: Cesar M. Diaz, Rancho Santa Margarita, CA (US); Peter Accorti, Jacksonville, FL (US)

Correspondence Address: Charles D. Gunter, Jr.;Whitaker, Chalk, Swindle & Sawyer, LLP, Suite 3500, 301 Commerce Street, Fort Worth, TX, 76102-4186, US

(21) Appl. No.: 11/363,361

(22) Filed: Feb. 27, 2006

Related U.S. Application Data
(63) Continuation-in-part of application No. 10/757,948, filed on Jan. 14, 2004, now abandoned.

(51) Int. Cl. A61N 1/39

(52) U.S. Cl. 607/5

(57) ABSTRACT

The present invention is a system for terminating cardiac arrhythmia using existing defibrillators found in the field in conjunction with a safe junction box. The system is designed to allow the defibrillator to connect to specially designed catheters equipped with specially designed electrodes and external electrodes for coupling energy to the heart that is greatly less than that used with external defibrillation alone. The system has the ability to create internal cardioversion vectors and also “hybrid” cardioversion vectors by allowing the external and internal electrodes to act together in the cardioversion process that is used to terminate arrhythmia in temporary and quasi-permanent implant applications. The quasi-permanent implant applications are greater than thirty day but less than twelve month applications where an implanted defibrillator may not be the ideal solution for patient care.

### CROSS REFERENCE TO RELATED APPLICATIONS

The present application is a continuation-in-part of earlier filed Ser. No. 10/757,948, filed Jan. 14, 2004, entitled “A System For Terminating Heart Arrhythmia Using Electrical Shocks Delivered Through A Set Of Internal And External Electrodes Configurable To A Multitude Of Shock Configurations By Selecting The Shock Vector(s) On A Control Device That Also Provides Over Shock Safety For Patients”, by the same inventors.

### BACKGROUND OF THE INVENTION

1. Field of the Invention

The present invention generally relates to cardioversion apparatus and methods and more particularly to a system for terminating heart arrhythmias using electrical shocks delivered through a set of electrodes usable in a variety of configurations for producing electric field vectors in the heart tissues of a patient.

2. Description of the Prior Art

Recent statistics indicate that approximately 3-4 million Americans will undergo Electrophyisology studies this year and that this number is expected to rise dramatically because of the aging population. The expected rise is increased further with the new scientific understanding of the impact that atrial fibrillation and atrial flutter have on the cause or aggravation of other diseases such as stroke and of heart failure. As a result, additional measures are needed to help save lives but also to eliminate life debilitating injury, such as that caused by stroke, early stages of heart failure and reduced physical exertion capacity caused by atrial fibrillation in which approximately 25% of the hearts blood pumping capacity is lost. The art and science of electrical cardioversion is not new nor is the use of transvenouse leads or patches rather than paddles for delivering the electrical energy to cardiovert. Early teachings can be found in patents by Stoft et al. (U.S. Pat. No. 3,566,876, issued March 1971); Jaros et al (U.S. Pat. No. 3,605,754, issued September 1971); Mirowski et al. (U.S. Pat. No. 3,614,955, issued October 1971 and U.S. Pat. No. 3,942,536, issued March 1976); Heilman et al (U.S. Pat. No. 4,270,549, issued June 1981) and others. Therefore, the generic methodology and/or apparatus art work can be referenced when considering systems that are designed to deliver electrical energy for cardioversion using paddles or patches or leads in combination with subcutaneous patches or wire meshes. However, little work has been done using a clinically viable and relevant system that allows for easy coupling to existing in-hospital defibrillators or a defibrillator that is designed and equipped to deliver low energies on demand separate from the rescue shock high energy external defibrillation.

Levy used a catheter to a metal plate shock vector for terminating chronic atrial fibrillation in 1998 and published his work; see, “High Energy Transcatheter Cardioversion Of Chronic Atrial Fibrillation”; Levy S, Lauribe P, Dolla; Circulation 1988; 12:514-8. Additional work published by Levy also compared internal cardioversion against external in a randomized trial that showed reduced energy levels with increased cardioversion success for internal cardioversion; see the published work titled “A Randomized Comparison of Internal and External Cardioversion of Chronic Atrial Fibrillation”, Levy S, Lacombe P, Cointe R, Bru P.; Journal of the American College of Cardiology 1992; 86; 1415-20. These referenced teachings defined a methodology of placing catheters in the right atrium and coronary sinus to form a vector for cardioversion suitable for cardioversion of atrial fibrillation.

Work published by Alt and others has continued to expand on the teaching of Levy with respect to using standard electro physiology-like catheters for the purpose of internal cardioversion. As a result, a step in the science of cardioversion electrodes was skipped or otherwise missed. Specifically, the art and science of cardioversion electrodes and catheter properties required to make a device clinically useful, safe and effective was not adequately addressed. For example, the connection of diagnostic catheters to a field defibrillator designed to deliver as much as 400 Joules is possible with a basic cable. However, if the user accidentally uses an energy that is not proper for the device and with proper calculations for current densities, system impedance, and energy dispersion, the result can be burning and ablation of the patient's tissue.

### SUMMARY OF THE INVENTION

It is an object of the present invention is to provide a device and method which will terminate arrhythmia of the heart using electrical energy coupled between at least two electrodes, ideally designed to carry cardioversion energy and located within the body or located inside and outside of the body.

Another object of the invention is to provide a special junction box that is selectable to varied configurations including a normal external shock configuration for rescue shock back-up in the event a catheter is inadvertently moved.

Another object of the present invention is to teach the varied vectors that can be achieved using the junction box and catheter system of the invention with multiple electrodes, or with several catheters and/or single patch electrodes.

Another object of the invention is to provide an improved cardioversion apparatus and method of the above type having patient protective circuitry built into the junction box that limits the amount of energy the patient can receive via the internal electrodes when an accidental high energy shock is induced by the defibrillator.

In the method of cardioversion of the invention, a first and a second flexible, internal catheter are positioned at first and second predetermined locations defined with respect to a mammalian heart. A predetermined output signal generated by a defibrillator is coupled through a passive control unit having at least first and second selectable output modes to the at least first and second flexible catheters. At least one of the at least first and second output modes of the control unit is selected. The predetermined output signal is then applied to the first and second internal catheters, whereby at least one of a plurality of electromagnetic field vectors is generated in selected tissue structures of a patient for producing a cardioversion effect therein. The mammalian heart can be, for example, a human heart having first and second atrial chambers and first and second ventricular chambers.

In one version of the invention, the passive control unit includes at least a first input terminal and at least a first pair of output terminals. A selector switch is coupled between the first input and the first output terminals. The selector switch has at least the first and second output modes for directing defibrillation energy to the first and second internal catheters. An energy absorbing device having a predetermined threshold is coupled from the first input terminal to a return conductor.

Preferably, at least one output mode provides an OFF condition of the control unit corresponding to a pass-through configuration. At least one output mode provides a defibrillation signal to the first and second internal catheters. Alternatively, at least one output mode provides a defibrillation signal to first and second external conduction pads. Additionally, the at least one output mode can provide a defibrillation signal to at least one combination of an internal catheter and an external conduction pad. The electromagnetic field which is created can comprise an electric field (“E field”) established by a predetermined current density within at least one internal catheter.

A passive energy limiting control unit for cardioversion is also provided for use with a defibrillator unit. The control unit includes a first selector circuit having an input terminal coupled to a switch common terminal, a plurality of output terminals connected to a respective plurality of switch terminals, and a movable contact for selecting a connection between the common terminal and a selected one of the plurality of output terminals. A return circuit is provided which is common to the input terminal and the plurality of output terminals. An energy absorbing device having a threshold rating is coupled between the input terminal and the return circuit. The threshold rating limits energy delivered to the output terminals. In one version of the control unit, the selector circuit is a rotary switch and the energy absorbing device is a metal oxide varistor. A second selector circuit can also be coupled in tandem with the first selector circuit.

A system is also provided for adapting a defibrillator unit to cardioversion therapeutic uses. The system includes a passive energy limiting control unit having an input for coupling an output of a defibrillator unit. At least first and second flexible catheter probes are provided for cardioversion. First and second cables connect the first and second catheter probes to respective output terminals of the energy limiting control unit. The energy limiting control unit comprises a first selector circuit having an input terminal coupled to a switch common terminal, a plurality of output terminals connected to a respective plurality of switch terminals, and a movable contact for selecting a connection between the common terminal and a selected one of the plurality of output terminals. A return circuit is common to the input terminal and the plurality of output terminals. An energy absorbing device having a threshold rating is coupled between the input terminal and the return circuit. A second selector circuit can also be coupled in tandem with a first selector circuit.

Additional objects, features and advantages will be apparent in the written description which follows.

### BRIEF DESCRIPTION OF THE DRAWINGS

FIG. 1 is one possible external configuration of the control unit of the invention.

FIGS. 2-12 are simplified schematic views of the various shock vector configurations which are generated between internal and/or external electrodes using the device and method of the invention.

FIG. 13 is a schematic view of one arrangement of the components of the system of the invention in place on a patient's body.

FIG. 14 illustrates one embodiment of a control circuit for use in the practice of the present invention.

### DETAILED DESCRIPTION OF THE INVENTION

The present invention, in part, comprises a vector selectable junction box with patient protective circuitry that couples to standard field external defibrillators. A special “safe junction box” provides a circuit that is activated once an unsafe energy is reached and redirects the excess energy away from the patient as a safety measure. The term “catheter mounted cardioversion electrode” as used in the following discussion describes an electrode that has an ideal electrode surface in which less than 2 Amps per centimeter squared is achieved by the design of the electrode and the protective circuitry within the junction or switch box.

The external cutaneous “patch” electrode is another term to be used in the following discussion and ideally describes an electrode as commonly known in the art of external defibrillation. This type of patch is ideally designed for high-energy external cardioversion (>50 Joules) but is also capable of coupling to low energy.

The safe junction box of the invention, in one preferred form, comprises a high performance and high durability switch designed to switch energy as low as 0.1 milli Volts up to 10,000 volts and selectable to 3 optional circuit paths. The switch must be designed to comply with all UL and IEC-601 requirements but must be ideally designed not increase resistance and or impedance to defibrillation voltage coupled from an external defibrillator.

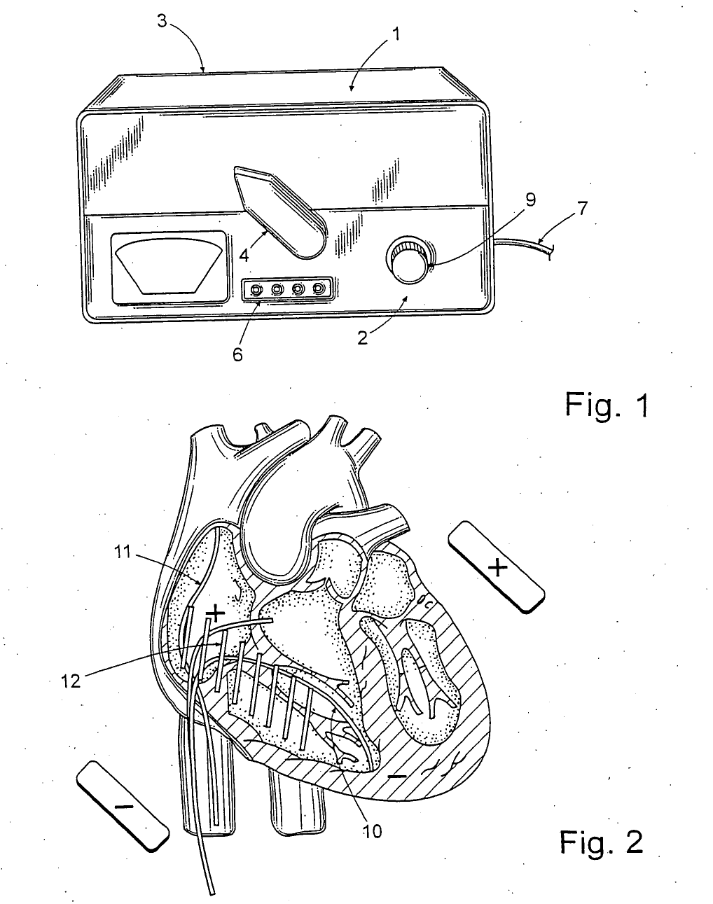

The apparatus of the invention will first be described with reference to FIGS. 1 and 2. FIG. 1 shows one preferred embodiment of the safe junction box used in the practice of the invention. The switch box consists of an electrically isolated housing 1 with electrically isolated face plate 2 and electrically isolated back plate 3 that forms the isolated casing. The safe junction box includes a high voltage switch 4 selectable to three positions that directs energy from the defibrillator through the patient protective circuitry or away from the patient protective circuitry when external (back-up) defibrillation is desired. The internal workings of the switch box are designed with semi-conductor technology which is applied to a high voltage application that “passively” senses voltages so that they do not exceed 1000 VDC. In the case an excessive voltage is sensed, the circuitry diverts the balance of voltage to a secondary path that absorbs the voltage. The circuit is not designed to stop unsafe voltage but is designed to limit the amount of voltage delivered to the patient. The patient always receives the energy required for cardioversion but protective circuitry controls the maximum amount of energy delivered, so that electrode heating does not occur at the surface of the patient's body, otherwise known and as ablation. The safe junction box can change the desired clinical result by either controlling voltage for safe cardioversion when an electrode is located within the heart and also for high voltage (unprotected side of circuit) for external cardioversion. Additionally, a defibrillator input connector 6 is mounted on the switch box so that any external defibrillator can be connected by using an adaptor cable (not shown) for each of the brands available in the field in any specific region of the world.

The energy coupled into the safe junction box through the input connector 6 is directed by means of the position of the switch 4 to either a protected circuit or unprotected circuit that allows energy from a defibrillator to pass unmodified. The energy from either the protected circuit or unprotected circuit is directed to connectors found on the back 3 or front plate 2 of switch box for catheter electrode connections or cutaneous patch or paddle (external electrode) connection 9.

FIG. 2 illustrates one possible embodiment of the present design that has three selections that have two distinct high voltage shock vector configurations. The first configuration is a shock between two internal electrodes that are in direct contact with the myocardium. The second setting is configured so that a bi-directional shock vector is obtained by directing current flow to one of the internal electrodes and also to one of the external (located outside the body) electrodes. The third configuration is an external only shock configuration, whereby the two external electrodes allow current flow between them so as to allow standard “life saving” defibrillation or cardioversion therapy to be applied.

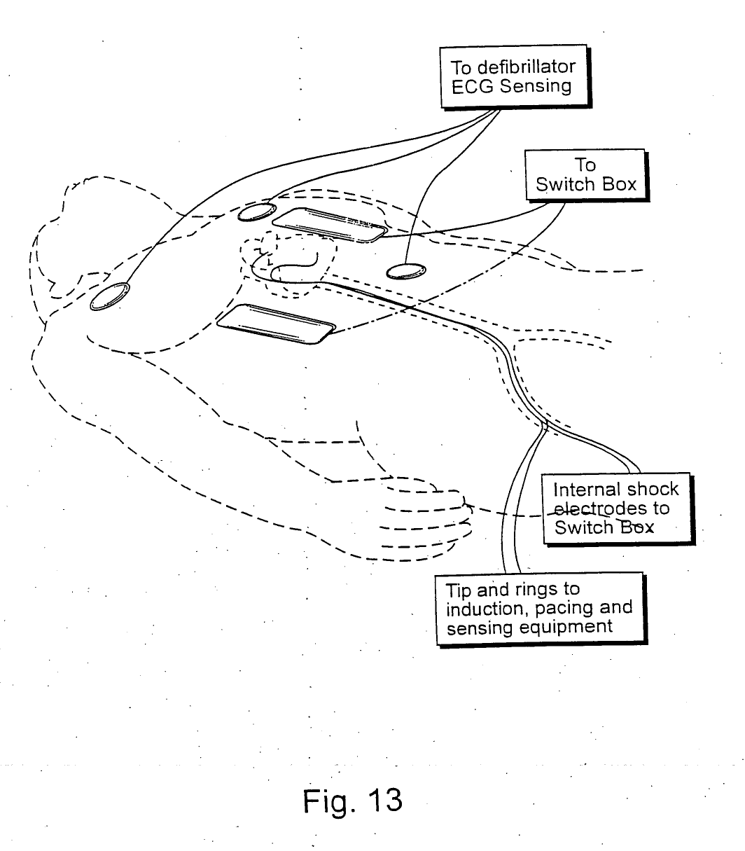

The first and second switch positions have protective circuitry that eliminates the possibility of energy greater than a predetermined threshold (1200 VDC) to be delivered to patient when any internal electrode is active. The third selection has no effect at all on the amount of energy delivered and only acts as a “selectable” passive conduit to the patient so that very high energy (>1200 VDC) shocks can be delivered between two external electrodes. The two external electrodes are preferably positioned in the posterior and anterior position as shown in FIG. 13 with anterior electrode preferable being the active external electrode when used in combination with internal electrode(s). However, both external electrodes can also be made to be active, as will be apparent to those skilled in the relevant arts. The system would include any cables and/or test devices required to effectuate the connection, pre-testing and or clinical utility as is know in the art or specific to the system herein disclosed.

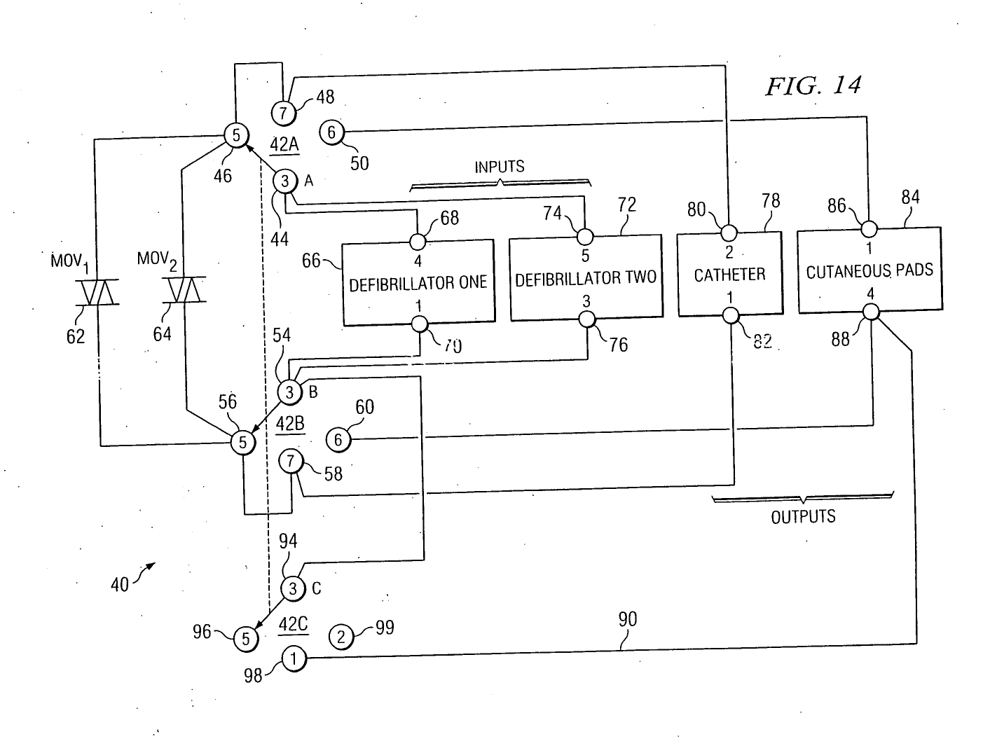

Referring to FIG. 14, there is illustrated one embodiment of a circuit diagram for a control circuit according to the present invention. The control circuit 40 includes a three-section rotary switch 42 having a first section 42A, a second section 42B, and a third section 42C disposed on a common shaft (not shown in this schematic diagram). The wiper of each section 42A, 42B, and 42C of the switch is connected to pin 3 (44) on section 42A, pin 3 (54) on section 42B, and pin 3 (94) on section 42C. The rotary switch 42 is configured as a selector to select which of three possible sets of output connections are to be provided to one set of input connections. The three sets of output connections are connected to terminals 5, 7 and 6 of the rotary switch 42 in a clockwise sequence as shown in the drawing. The set of inputs are connected to terminal 3 (44, 54, 94) of each section of the rotary switch 42.

Terminal 5 (46) of the first section 42A of the rotary switch 42 is connected to a first lead of a first metal oxide varistor (MOV) 62 and to a first lead of a second MOV 64. Terminal 5 (54) of the second section 42B of the rotary switch 42 is connected to a second lead of each first 62 and second 64 MOV. As is well-known, an MOV is a semiconducting device for absorbing energy from a voltage signal impressed across it in excess of a rated voltage level. In the illustrative embodiment, the voltage rating may be 1000 Volts and the energy-absorption capacity of each MOV 62, 64 may be specified at 25 Joules, providing a total energy absorbing capacity of 50 Joules. Thus, if energy in excess of 50 Joules is applied to an output of the control circuit, the excess energy will be absorbed by the first and second MOVs 62, 64.

Continuing with FIG. 14, connectors for two defibrillator inputs are provided, both connected to terminals 3 (44, 54) of the rotary switch 42. A first terminal 68 of the first defibrillator connector 66 is connected to pin 3 (44) of rotary switch 42. A second terminal 70 of the first defibrillator connector 66 is connected to pin 3 (54) of rotary switch 42. Similarly, a first terminal 74 of the second defibrillator connector 72 is connected to pin 3 (44) of rotary switch 42, and a second terminal 76 of the second defibrillator connector 76 is connected to pin 3 (54) of rotary switch 42. The first 66 and second 72 defibrillator connectors provide for connecting defibrillator units having different connecting cables.

Terminals 5 (46, 56) of the respective first 42A and second 42B sections of the rotary switch 42 are connected respectively to terminals 7 (48, 58) of the first 42A and second 42B sections of the rotary switch 42. Terminals 7 (48, 58) are connected to respective first 80 and second 82 terminals of a first output connector 78. This connector 78 is configured to connect with a cable attached to a catheter (not shown), which is an internal electrode connection of the equipment to a patient when the system is in use. Terminals 6 (50, 60) of the respective first 42A and second 42B sections of the rotary switch 42 are connected respectively to the first 86 and second 88 terminals of a second output connector 84. This second output connector 84 is configured to connect with a cable attached to cutaneous pads (not shown), which provide an external electrode connection of the equipment with the patient.

The third section 42C of the rotary switch 42 provides for connecting the defibrillator unit input to one internal catheter electrode and one external cutaneous pad. This may also be referred to as an “Internal+Patch” connection or mode. The third section 42C includes a terminal 98 in the center one of three positions that is connected to terminal 88 of the cutaneous pads 84 via a wire 90. This defibrillator connection is provided by connecting pin 3 (54) of the rotary switch 42 section B (42B) to pin 3 (94) of the rotary switch 42 section C (42C). There are no connections to pin 5 (96) and 2 (99) of section C (42C) of the rotary switch 42.

In operation, with a defibrillator (not shown) connected to either of the first 66 or second 72 input connectors, rotating the knob of the switch 4 of FIG. 1 to one of the three positions of the rotary switch 42 provides a selection of one of the three possible output combinations, respectively, INTERNAL (catheter), INTERNAL (Catheter+Patch), or EXTERNAL (Cutaneous pads), as may be designated in sequence on the front panel of the housing 1. These output selections enable application of a variety of combinations of cardioversion vectors to the patient under treatment.

FIGS. 2-12 are simplified schematic views of the various shock vector configurations which are generated between internal and/or external electrodes using the device and method of the invention.

In FIG. 2, one of the preferred embodiments of the invention is shown in the “as used” setting in the heart. A catheter having a high surface electrode 10 is located in right ventricle and a second catheter having a high surface electrode 11 is located right atrium. The energy provided by an external defibrillator forms a single dimension vector 12 that is ideally suited to terminate ventricular fibrillation or ventricular tachycardia.

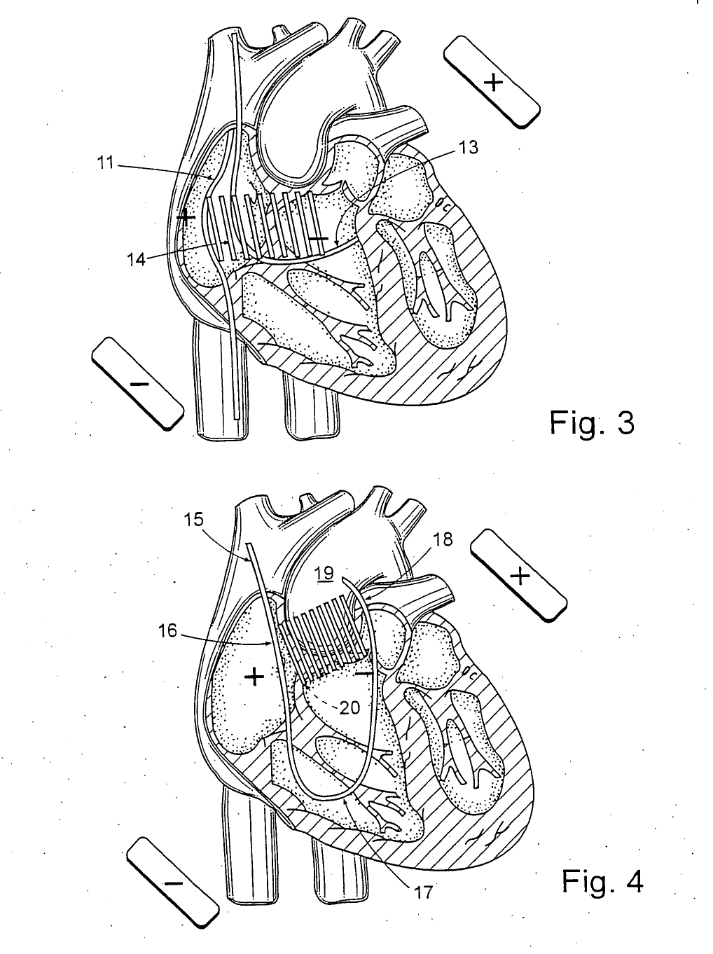

In FIG. 3 another preferred embodiment is shown in the “as used” setting in the heart. A catheter having a high surface electrode 11 is located in the right atrium and a second high surface electrode 13 is located in the coronary sinus. The energy provided by an external defibrillator forms a single dimension vector 14 that is ideally suited to terminate atrial fibrillation, atrial flutter and atrial tachycardia.

In FIG. 4 another preferred embodiment of the invention is shown in the “as used” setting in the heart. A catheter having 3 high surface electrodes 15 is positioned so that one electrode is located in right atrium 16, a second high surface electrode is located in the right ventricle 17 and a third is located in the pulmonary artery 18. The energy provided by an external defibrillator forms a single dimension vector between 16 and 18, resulting in a single dimension vector 19 that is ideally suited to terminate atrial fibrillation, atrial flutter and atrial tachycardia. Alternatively in a single dimension vector formed between 16 and 17 is ideally suited to terminated ventricular fibrillation or ventricular tachycardia. All of these actions are accomplished by using a single catheter that triangulates the heart in a “quasi-Ivanhoe” triangle configuration.

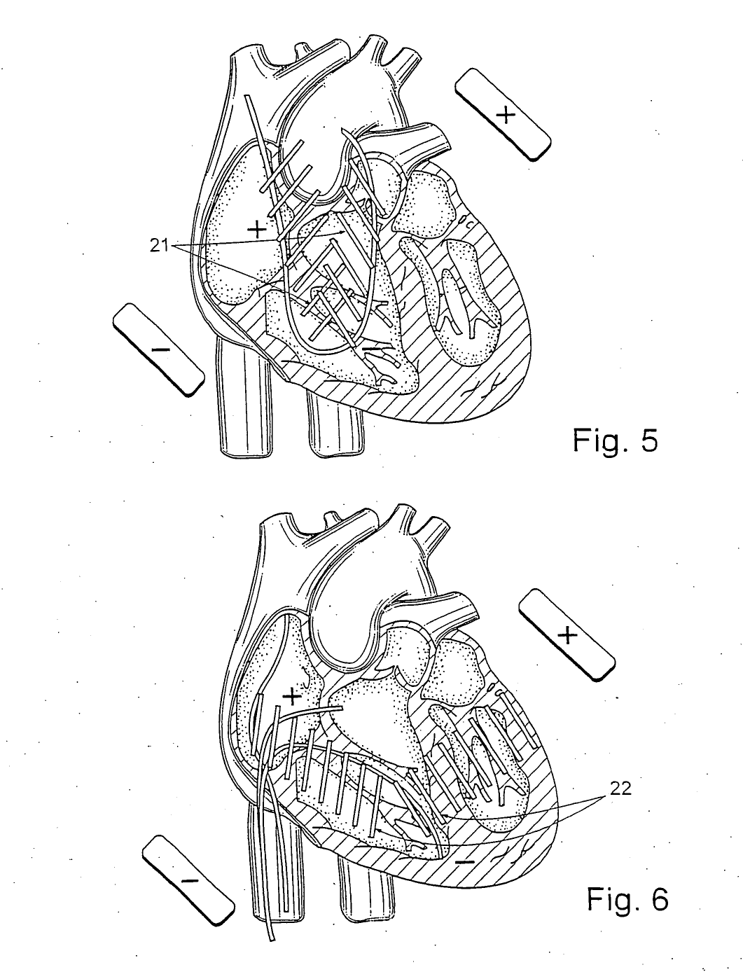

FIG. 5 is similar to FIG. 4, except that a two-dimensional shock 21 that is ideal for terminating ventricular fibrillation or ventricular tachycardia is created. FIG. 5 shows an internal only shock configuration, through the switch box (protected circuit), whereby the shock vector is between three electrodes on a single catheter having electrodes located in the pulmonary artery, right atrium and right ventricle and whereby the resulting shock vector is bi-directional. Specifically, one electrode provides the source of current while the other two electrodes comprise return paths, or two electrodes provide a source of current and one electrode becomes a return path.

FIG. 6 is similar to FIG. 2 except that a two dimension shock 22 is created which is ideal for terminating ventricular fibrillation or ventricular tachycardia. FIG. 6 shows a shock configuration, through the switch box (protected circuit), whereby the shock vector produced is between the catheter in the right ventricle and the catheter in the right atrium and the paddle (gel pad) located on the patients chest. The shock is therefore bi-directional and vectors between one internal electrode to the other internal electrode plus the external electrode provide one source of current and two return paths in two very different planes that form vector paths of 15 to 90 degrees difference between vectors. The increased separation of the shock vectors improves the amount of myocardium captured and stimulated causes it to function normally (sinus rhythm due to cardioversion or defibrillation).

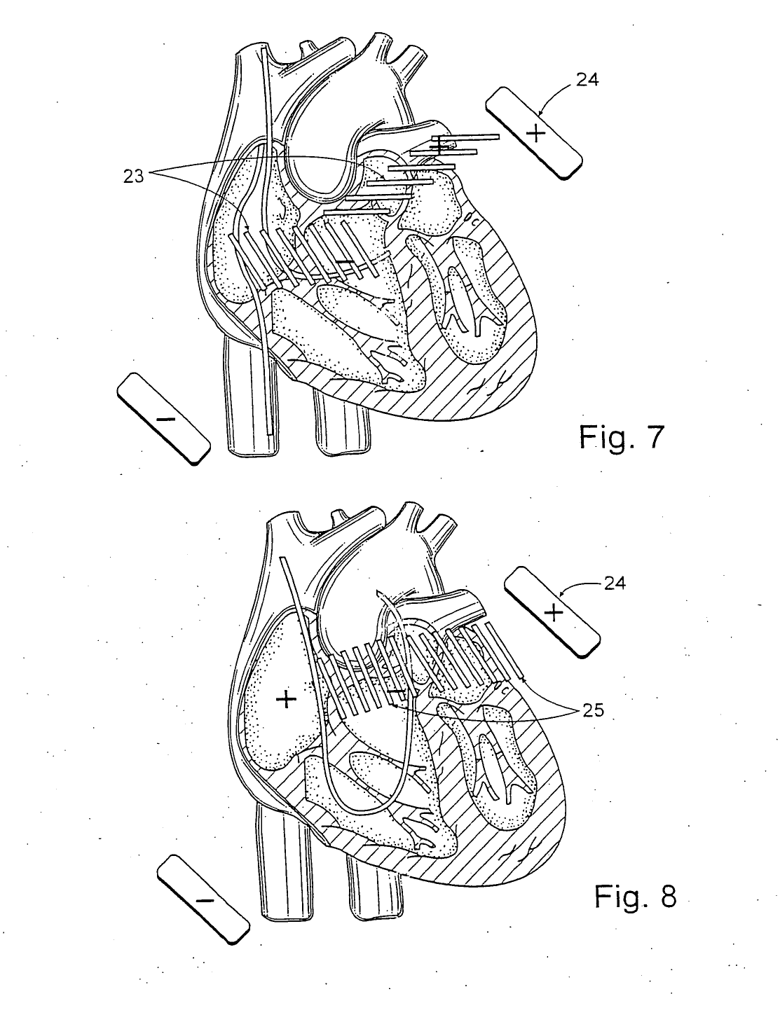

FIG. 7 is similar to FIG. 3, except that a two dimension shock 23 is created which is ideal for terminating atrial fibrillation, atrial flutter and atrial tachycardia, because energy captures the left atria. The cutaneous patch 24 is made active via the switch box yet the other patch remains inactive so that the energy vector is formulated by the operation of the safe junction box. In the embodiment of the invention shown in FIG. 7, the shock vector is between the catheter in the coronary sinus and the right atrium and also the paddle (gel pad) located on the patient's chest. The shock is therefore bi-directional and vectors between one internal electrode to the other internal electrode plus the external electrode provide one source of current and two return paths in two very different planes that form vector paths of 15 to 90 degrees difference between vectors. The increased separation of the shock vectors improves the amount of myocardium captured and the resulting stimulation causes it to function normally (sinus rhythm due to cardioversion or defibrillation). The use of the coronary sinus for one vector provides for more of the atria (both the left and right atrium) to be captured when using this configuration.

FIG. 8 is similar to FIG. 4, except that a two dimension shock 25 is created which is ideal for terminating atrial fibrillation, atrial flutter and atrial tachycardia because energy being generated captures the left atria. The cutaneous patch 24 is made active via the switch box yet the other patch remains inactive so that the energy vector is formulated by the safe junction box.

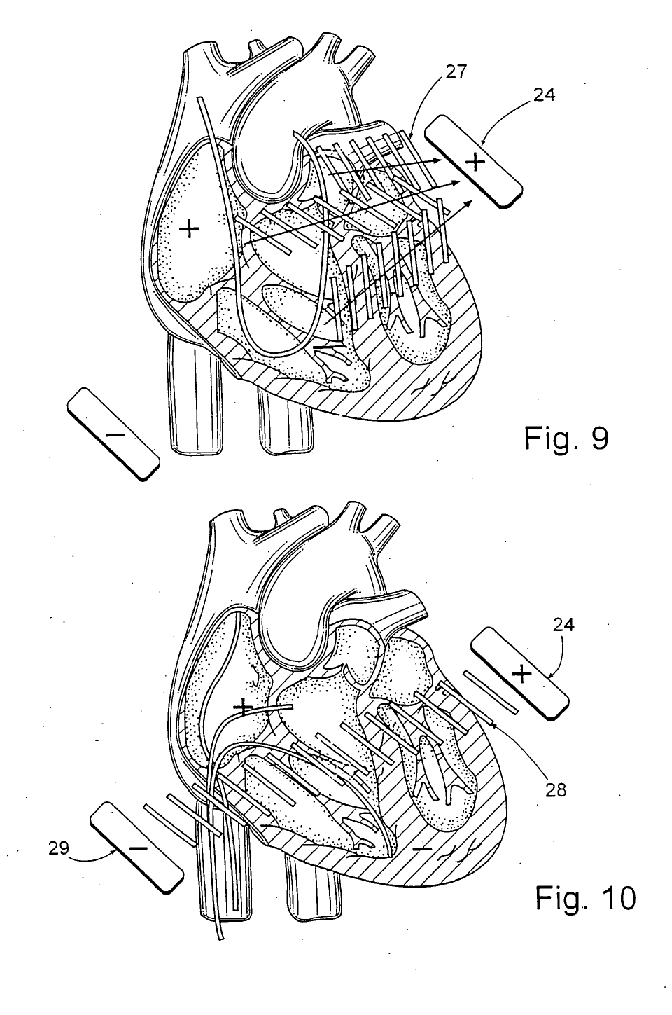

FIG. 9 is similar to FIG. 4, except that a three dimensional shock 25 is created which is ideal for terminating all arrhythmia because the energy captures all four chambers of the heart and primarily the left side of heart. The cutaneous patch 24 is made active via the switch box yet the other patch remains inactive so that the three dimensional energy vector is formulated by the safe junction box. The catheter electrodes are all made active and of the same polarity with only a single cutaneous patch being made the opposite polarity, with the result that a three dimensional vector is formed.

FIG. 10 shows how the energy can be converted back to standard external defibrillation mode when the switch 4 is turned to the proper location. A high energy vector is created when the cutaneous patch 24 is made active with one polarity and the other cutaneous patch 29 is made active with opposite polarity, to thereby form a “standard” external defibrillation shock vector 28. FIG. 10 thus shows an external only shock configuration, through the switch box (passive unprotected circuit side) that allows the delivery of high energy through paddles or gel pads.

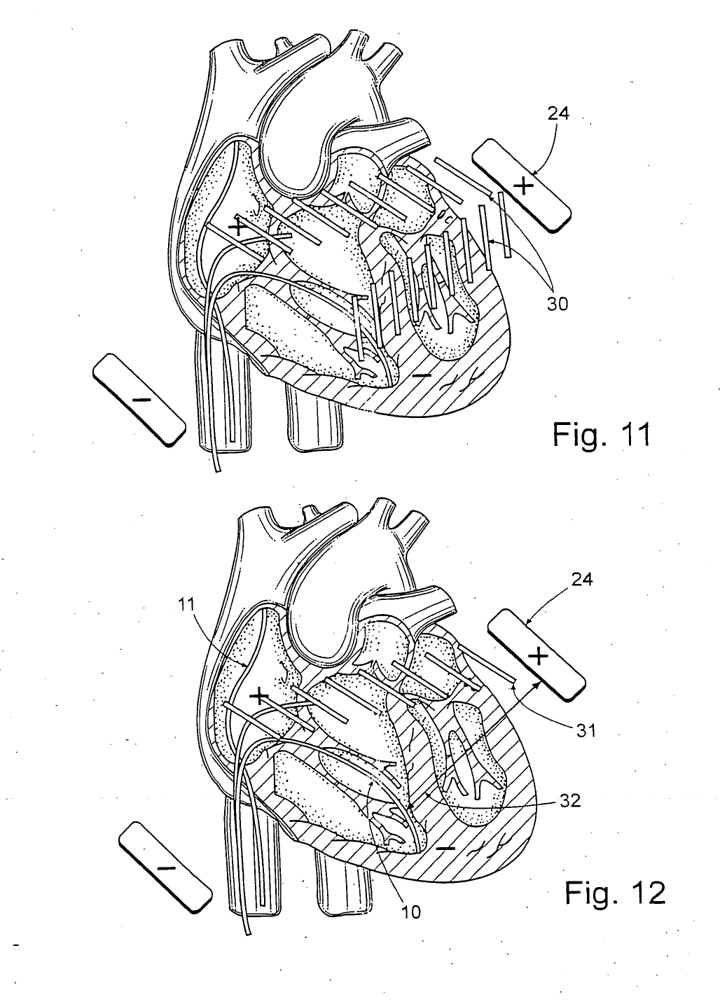

FIG. 11 is similar to FIG. 2, except that two single dimension shocks with non-uniform alignment 30 are created which are ideal for terminating any arrhythmia of the heart. In FIG. 11, the shock vectors are between the catheters in the right atrium and the right ventricle and propagate thru the heart to the paddle (gel pad) located on the patient's chest. The shock therefore has two distinct vector angles formed between two internal electrodes and one or more external electrodes. The configuration is ideally suited to capture large ventricular and atrial mass for terminating VT, VF and SVT's during EP studies and ablation procedures.

FIG. 12 is similar to FIG. 2, except that a one single dimension shock with non-uniform alignment 31 is created which is ideal for terminating atrial fibrillation, atrial flutter and atrial tachycardia, because energy captures the left atria. The shock vector travels through the entire heart muscle since it is trying to couple to the cutaneous patch through the outer wall of the left atria. Additionally, FIG. 12 teaches by simplified pictorial description, that a catheter in the right atrium can be made inactive and that a catheter in left ventricle can be made electrically active, whereby a single dimensional vector 32 can be created that is ideal for terminating ventricular fibrillation or ventricular tachycardia. The shock vectors are between the catheters in the right atrium and propagate thru the heart to the paddle (gel pad) located on the patient's chest. The shock therefore has one distinct vector angle formed between the internal electrode and one or more external electrodes. The configuration is ideally suited to capture large atrial mass for terminating AF during EP studies and ablation procedures and for easily and safely terminating chronic refractory atrial fibrillation (AF that has failed cardioversion using drugs and external cardioversion).

The technique or method taught in FIG. 12 could be used with only one electrode in the right atrium (without having a catheter in right ventricle) coupled to a single external electrode only that would form an energy vector through both atria. The ECG electrodes on the chest of patient would be used to synchronize shock with QRS complex. Additionally, the use of low energy (<50 J) means that anesthesia and intubation is not required. Deep sedation and/or conscious sedation can be utilized which reduces the need for specialized staff to conduct what should be a routine procedure.

FIG. 13 is a schematic representation of one possible “as used” configuration of the system of the invention that utilizes EKG pads directly from the defibrillator to synchronize the shock for any tachycardia, fibrillation or flutter. A cutaneous electrode (patch, paddle or similar device) is located on the back of the patient 13 and a second cutaneous electrode (patch, paddle or similar device) is located on the chest of the patient 14. The cutaneous electrode located on the chest is made active by the safe junction box and also the high surface area electrodes located on the heart.

An invention has been provided with several advantages. The device and method of the invention can be used effectively to terminate arrhythmia of the heart using electrical energy coupled between at least two electrodes. The system is designed to carry cardioversion energy and can be located within the body or located inside and outside of the body. The special junction box provided as a part of the system is selectable to varied configurations including a normal external shock configuration for rescue shock back-up in the event a catheter is inadvertently moved. Varied vectors can be achieved using the junction box and catheter system of the invention with multiple electrodes, or with several catheters and/or single patch electrodes. The junction box features patient protective circuitry that limits the amount of energy the patient can receive via the internal electrodes when an accidental high energy shock is induced by the defibrillator.

While the invention has been shown in several of its forms, it is not thus limited but is susceptible to various changes and modifications without departing from the spirit thereof.

What is claimed is:

1. A method of cardioversion, comprising the steps of:
positioning a first and a second flexible, internal catheter respectively at first and second predetermined locations defined with respect to a mammalian heart;
coupling a predetermined output signal generated by a defibrillator through a passive control unit having at least first and second selectable output modes to the at least first and second flexible catheters;
selecting at least one of the at least first and second output modes of the control unit;
applying the predetermined output signal to the first and second internal catheters; and
generating at least one of a plurality of electromagnetic field vectors in selected tissue structures of a patient for producing a cardioversion effect therein.

2. The method of claim 1, wherein the mammalian heart is a human heart having first and second atrial chambers and first and second ventricular chambers.

3. The method of claim 1, wherein the passive control unit comprises:
at least a first input terminal;
at least a first pair of output terminals;
a selector switch coupled between the first input and the first output terminals and having at least the first and second output modes for directing defibrillation energy to the first and second internal catheters; and
an energy absorbing device having a predetermined threshold coupled from the first input terminal to a return conductor.

4. The method of claim 1, wherein at least one output mode provides an OFF condition of the control unit corresponding to a pass-through configuration.

5. The method of claim 1, wherein at least one output mode provides a defibrillation signal to the first and second internal catheters.

6. The method of claim 1, wherein at least one output mode provides a defibrillation signal to first and second external conduction pads.

7. The method of claim 1, wherein at least one output mode provides a defibrillation signal to at least one combination of an internal catheter and an external conduction pad.

8. The method of claim 1, wherein the electromagnetic field comprises an electric field (“E field”) established by a predetermined current density within at least one internal catheter.

9. A passive energy limiting control unit for cardioversion for use with a defibrillator unit, comprising:
a first selector circuit having an input terminal coupled to a switch common terminal, a plurality of output terminals connected to a respective plurality of switch terminals, and a movable contact for selecting a connection between the common terminal and a selected one of the plurality of output terminals;
a return circuit common to the input terminal and the plurality of output terminals; and
an energy absorbing device having a threshold rating and coupled between the input terminal and the return circuit.

10. The control unit of claim 9, wherein the selector circuit is a rotary switch.

11. The control unit of claim 9, wherein the threshold rating limits energy delivered to the output terminals.

12. The control unit of claim 9, wherein the energy absorbing device is a metal oxide varistor.

13. The control unit of claim 9, wherein further comprising at least a second selector circuit coupled in tandem with a first selector circuit.

14. A system for adapting a defibrillator unit to cardioversion therapeutic uses, comprising:
a passive energy limiting control unit having an input for coupling an output of a defibrillator unit;
at least first and second flexible catheter probes for cardioversion; and
first and second cables connecting the first and second catheter probes to respective output terminals of the energy limiting control unit.

15. The system of claim 14, wherein the energy limiting control unit comprises:
a first selector circuit having an input terminal coupled to a switch common terminal, a plurality of output terminals connected to a respective plurality of switch terminals, and a movable contact for selecting a connection between the common terminal and a selected one of the plurality of output terminals;
a return circuit common to the input terminal and the plurality of output terminals; and
an energy absorbing device having a threshold rating and coupled between the input terminal and the return circuit.

16. The control unit of claim 15, further comprising at least a second selector circuit coupled in tandem with a first selector circuit.
